(19) United States
(12) Patent Application Publication
(10) Pub. No.: US 2022/0125987 A1
(43) Pub. Date: Apr. 28, 2022

(54) WOUND CLOSURE DEVICES COMPRISING PROTOCATECHUIC ACID

(71) Applicant: Lanny Leo Johnson, Frankfort, MI (US)

(72) Inventor: Lanny Leo Johnson, Frankfort, MI (US)

(21) Appl. No.: 17/078,892

(22) Filed: Oct. 23, 2020

(51) Int. Cl. A61L 17/08; A61L 17/14; A61L 17/04; A61L 17/10; A61L 17/12

(52) U.S. Cl. CPC A61L 17/08; A61L 17/145; A61L 2420/02; A61L 17/105; A61L 17/12; A61L 17/04

(57) ABSTRACT

A suture or a surgical or wound closure staple is disclosed that includes protocatechuic acid. The protocatechuic acid may be coated on, or impregnated in, the suture or wound closure staple. The suture or wound closure staple may include polypropylene, nylon, polyester, and/or braided polyester, catgut, 85/15 D,L lactide/glycolide, and/or 910 Vicryl. The protocatechuic acid may coat 25% or more of the surface of the suture or surgical staple. In embodiments, the protocatechuic acid may have a purity of 95% or greater. The protocatechuic acid may include crystalline protocatechuic acid.

### BACKGROUND OF THE DISCLOSURE

### Field of the Invention

The present disclosure is generally directed to sutures and surgical staples, and more specifically, sutures and surgical staples that comprise protocatechuic acid preferably either coated or impregnated therein.

### Description of the Related Art

Sutures and surgical or wound closure staples are considered, for example, by the Food and Drug Administration (FDA) as implants. As such, like other implants, they present the risk of bringing microbes into a wound and thereby creating an infection. Current state-of-the-art provides an antibiotic impregnated suture on the market, Johnson & Johnson™ 910 bio-absorbable copolymer of 10 L lactide and 90 glycolide with Triclosan™ as the antibiotic.

Triclosan™, however, carries potential health concerns. This is due to possible antimicrobial resistance, endocrine disruption, and other issues. Triclosan™ has been designated as a contaminant of emerging concern (CEC) and is under investigation for public health risk. Triclosan™ is also suspected of potential adverse ecological and associated human health effects. Triclosan™ is also thought to accumulate in wastewater and return to drinking water, thus propagating a buildup that could cause increasing negative effects with ongoing use.

In the United States, after a decades-long review of the potential health issues from this contaminant of emerging concern, the FDA ruled on Sep. 6, 2016, that Triclosan™ was not generally recognized as safe and effective (GRAS/GRAE).

Triclosan™ has also been banned by the FDA for other uses including antibacterial soaps and body washes, toothpastes, and some cosmetics, all products regulated by the U.S. Food and Drug Administration. The commercially available Triclosan™ suture is also not used for neurological cases.

There is, accordingly, a need in the art for an improved, safe, non-toxic, antibiotic suture and/or staple.

### SUMMARY OF THE INVENTION

In embodiments wound closure devices including a suture or a surgical or wound closure staple, or related device, including protocatechuic acid are disclosed. The protocatechuic acid may be coated on, or impregnated in, the suture or wound closure staple. The suture or wound closure staple may include polypropylene, nylon, polyester, and/or braided polyester, catgut, 85/15 D,L lactide/glycolide, and/or 910 Vicryl.

The protocatechuic acid may coat 25% or more of the surface of the suture or surgical staple. In embodiments, the protocatechuic acid may coat 75% or more of the surface of the suture or wound closure staple. In embodiments, the protocatechuic acid may coat 95% or more of the surface of the suture or wound closure staple.

In embodiments, the protocatechuic acid may have a purity of 95% or greater. In embodiments, the protocatechuic acid may include crystalline protocatechuic acid.

In embodiments, a method of making a suture or wound closure staple including protocatechuic acid may include placing a suture or wound closure staple in contact with protocatechuic acid. In embodiments, a method of making a suture or wound closure staple including protocatechuic acid may include drawing the suture or wound closure staple through dry protocatechuic acid. In embodiments, the suture or wound closure staple may be drawn through dry protocatechuic acid crystals under pressure. In other embodiments, the suture or surgical staple may be impregnated with protocatechuic acid and/or protocatechuic acid crystals.

Other features and aspects will be apparent from the following detailed description, the drawings, and the claims

### BRIEF DESCRIPTION OF THE DRAWINGS

FIG. 1A shows a low power polarized photomicrograph of dried 0.9% sodium chloride on a glass slide.

FIG. 1B shows a low power polarized photomicrograph of dried PCA crystals on a glass slide.

FIG. 1C shows a high power polarized light photomicrograph of dried PCA and 0.9% normal saline.

FIG. 2A shows low power photomicrographs of salt on a polypropylene suture.

FIG. 2B shows coated 5-0 polypropylene suture with PCA crystals.

FIG. 2C shows photomicrographs showing PCA on 0 polypropylene suture under polarized light.

FIG. 3A shows a polarized photomicrograph of PCA crystals on 0 polypropylene suture.

FIG. 3B shows high power photomicrographs of PCA on 0 polypropylene suture.

FIG. 3C shows 1% PCA in alcohol on 5 0 polypropylene suture.

FIG. 4A shows high power polarized photomicrograph of crystal on 5-0 polypropylene from propylene glycol after 24 hours.

FIG. 4B shows different crystal shape and size on 5-0 polypropylene suture when in propylene glycol medium.

FIG. 4C shows photomicrograph low power of PCA material on the 0 nylon suture.

FIG. 5A shows higher power polarized light photomicrograph of PCA crystals attached.

FIG. 58 shows photomicrograph of few crystals when sprayed with 1% PCA in alcohol.

FIG. 6A shows higher power PCA crystals on 0 nylon with 20% in ethanol.

FIG. 6B shows low power photomicrographs with polarized light showing attached crystals to 0 nylon suture.

FIG. 6C shows higher power photomicrograph of polarized light with crystals on 5 0 nylon.

Throughout the drawings, the drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, products, and/or systems, described herein. However, various changes, modifications, and equivalents of the methods, products, and/or systems described herein will be apparent to an ordinary skilled artisan.

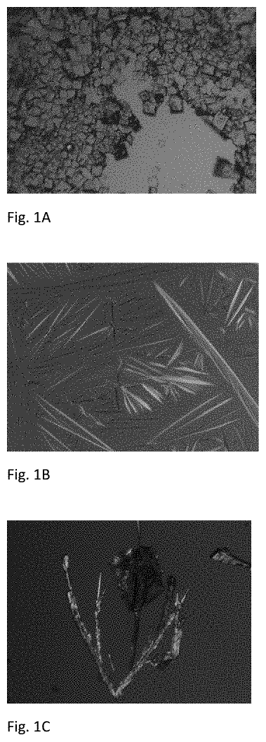

Protocatechuic acid is a broad-spectrum antibiotic and biofilm destroyer that can be used to coat or impregnate a suture or surgical staple either at the time of surgery or during manufacture. Previous testing has demonstrated that protocatechuic acid (PCA) is effective in killing a wide spectrum of bacteria and is useful for wound healing and the treatment of surfaces including medical devices, implants, etc., to reduce or eliminate bacteria. U.S. Pat. No. 10,004,705 contains test data demonstrating protocatechuic acid's effectiveness in this regard. See U.S. Pat. No. 10,004,705, Examples 1-11, columns 28-44, and FIGS. 1-43, which is hereby incorporated by reference. See also U.S. Pat. No. 9,925,152, Examples 1-6, and FIGS. 1-31, which is herein incorporated by reference.

Protocatechuic acid (PCA) (IUPAC: 3,4 dihydroxybenzoic acid) is found throughout nature; in the soil, and in plants. PCA is the primary metabolite from cyanidin-3-glucoside, a dye that makes blueberries blue and cherries red. PCA is common in the human diet in many vegetables and fruits. The human bowel bacteria manufacture small amounts daily. PCA upon ingestion perfuses all the cells and tissues of the human body in a matter of minutes. The entire metabolism is known with duration of eight hours prior to excretion in the urine and feces.

PCA is safe for human consumption. PCA has an existing FDA G.R.A.S. designation as Generally Recognized as Safe as a flavoring substance. Its FEMA number is 4430. PCA is non-toxic. There are no known allergy or mutagenic effects.

PCA is a powerful antioxidant, e.g., 10 times more powerful than vitamin E. Antioxidants are fundamental to health. PCA is a powerful anti-inflammatory reagent. Inflammation is known to be a common denominator of all disease. PCA enhanced the genetic expression in in vitro studies of local growth factors in human and rabbit synovium, rodent skin and human osteoblasts and mesenchymal stem cells to produce bone. There are known to be many, and varied, health benefits of protocatechuic acid.

PCA is nontoxic. Toxicity is greater than 5000 mg/kg body weight in female rats. The conversion to a human relative dose to exceed safety would be 350,000 milligrams per day for a 70-kilogram human. This amount is not likely to be ingested at once or even over a period of time.

The production methods for PCA are typically biochemical. The products are absent of trace metals. PCA is readily available in large amounts from several international manufacturers.

PCA may be a physical crystal retaining a crystalline condition in dry air as well as in a liquid vehicle or environment. The physical shape is one of sharp edges and projections, even shown to be needle-like in solution. The irregular sharp projections may physically disrupt a bacterial biofilm and the prongs and coating of SARS CoV2 upon contact. Compositions comprising PCA have been shown effective for antimicrobials and methods for wound healing. PCA is a biofilm destroyer for MRSA and Pseudomonas. The safety and effectiveness for controlling potential pathogens on human skin has also been demonstrated. There is no skin irritation.

PCA's Mode of Action may include multiple modes of action. Crystalline sharp shapes for disruption, low pH, anti-protease, docking blocking, enhancing the cellular and hormonal immunity, anti-tyrosinase, anti-thrombosis. Traditional antimicrobials function chemically or biochemically. Their biochemical inter-action disrupts the viral interaction with the host and/or physically disrupts the virus prongs or wall. Crystals by their physical nature have similar known cytotoxic properties.

Crystals may include atoms, ions, or biomolecules, and may cause tissue injury, inflammation, and re-modelling. This may be due to nucleation or crystal growth from a seed crystal formed on a surface medium, for example tubular epithelial cells, urolithiasis forming at Randall's plaques, calcifications in injured tendons, damaged cartilage or atheromatous vascular lesions, crystal formation itself causes tissue injury and inflammation, for example in gouty arthritis, pulmonary silicosis or asbestosis, cholesterol crystals driving atherogenesis and in oxalate, cystine or urate nephropathy. Crystals may also trigger tissue inflammation via the NLRP3 inflammasome and caspase-1-mediated secretion of IL-113 and IL-18. Crystals may also exert direct cytotoxic effects leading to necrotic rather than apoptotic cell death.

This shows that the physical properties of PCA crystals have an antiviral and antibacterial property, independent or in conjunction with their biochemical properties. That is, they can physically disrupt bacteria and virus integrity.

In some embodiments, the protocatechuic acid crystal is at least 80%, at least 90%, at least 95%, at least 98%, or at least 99% pure. In some embodiments, the protocatechuic acid crystal is essentially 100% pure. In some embodiments, the protocatechuic acid crystal is in particulate form. In some embodiments, the protocatechuic acid crystal is biochemically produced. In some embodiments, the protocatechuic acid crystal is produced by a plant extraction method. In some embodiments, the protocatechuic acid crystal contains trace metal content less than 100 ppm, less than 10 ppm, less than 1 ppm, less than 100 ppb, less than 10 ppb, or less than 1 ppb.

In general, wound closure devices as contemplated herein can include sutures, surgical staples, and a variety of glues and tapes which are used for wound closure purposes. Generally speaking, any wound closure device can be coated or impregnated with protocatechuic acid to achieve the beneficial effects described herein.

As used herein, a suture is a medical device used to hold body tissues together after an injury or surgery. Application generally involves using a needle with an attached length of thread. A number of different shapes, sizes, and thread materials are available. Surgeons, physicians, dentists, podiatrists, eye doctors, registered nurses and other trained nursing personnel, medics, clinical pharmacists, and veterinarians typically engage in suturing. Surgical knots are used to secure the sutures.

Suture thread can be made from numerous materials. Sutures can be made from biological materials, such as catgut suture and silk. Other materials include silver wire and synthetics, including the polyglycolic acid, polylactic acid, Monocryl and polydioxanone as well as nylon, polyester, PVDF, 85/15 D,L lactide/glycolide and 90 glycolide, and polypropylene. Sutures may come in specific sizes and may be either absorbable (naturally biodegradable in the body) or non-absorbable. Sutures must be strong enough to hold tissue securely but flexible enough to be knotted. They may be hypoallergenic.

Wound closure staples or surgical staples are specialized staples used in surgery in place of sutures to close skin wounds, connect or remove parts of the bowels or lungs. The use of staples over sutures may reduce local inflammatory response. In examples, clips instead of staples for some applications may also be used. Surgical staples may be made of titanium and stainless steel. Titanium may also be used. Synthetic staples may be based on polyglycolic acid, as well as any of the materials mentioned above for sutures.

As used herein impregnate means to saturate or infuse a material or to fill pores or spaces with a substance or material.

### Examples Showing PCA Coatings on Wound Closure Device Materials

Suture materials of various types were tested for PCA coating in dry, liquid and combination methods. The dry material was the PCA crystals. The liquid vehicles were normal saline, 1 and 20% PCA in ethanol, and propylene glycol with 15% PCA.

The types of material were nylon, polypropylene, and braided polyester. The sizes were 5-0 and 0. The braided polyester, 5-0 polypropylene and 0 nylon were tested.

One test method was to physically coat the suture material with dry crystals forced on the suture as it was pulled through a group of crystals held tightly within folded paper. The suture was inspected microscopically with regular and polarized light at two intervals: dry and after subjected to normal saline soaking. The latter to replicate the fluid environment of tissue.

The second method was to spray coat and or soak the suture material de novo in various concentrations of PCA in an alcohol vehicle. After drying the suture was inspected microscopically. Subsequent soaking in normal saline occurred prior to the second microscopic inspection.

To identify the physical shapes of the various crystals, photomicrographs were taken of saline and PC crystals alone and in combination. Photomicrographs were taken of the different suture material under various conditions; de novo, sprayed, impregnated and after subjected to normal saline environment.

### Results

The physical shapes of the various crystals under polarized light microscopy showed different shapes and varying response to polarized light.

Normal saline upon drying on a microscopic slide had square shaped crystals and no polarization as shown in FIG. 1A which shows a low power photomicrograph polarized of dried 0.9% sodium chloride on a glass slide.

Protocatechuic acid crystals were predominately needle shape in clumps. They were very reflective in polarized light as shown in FIG. 1B which shows a low power photomicrograph polarized of dried PCA crystals on a glass slide.

When PCA was combined with normal saline the resultant dried crystals showed a combination of the square and needle shapes often attached and with polarization as shown in FIG. 1C which shows a high power polarized light photomicrograph of dried PCA and 0.9% normal saline.

For polypropylene: 5-0, the first test was to coat the suture with normal saline.

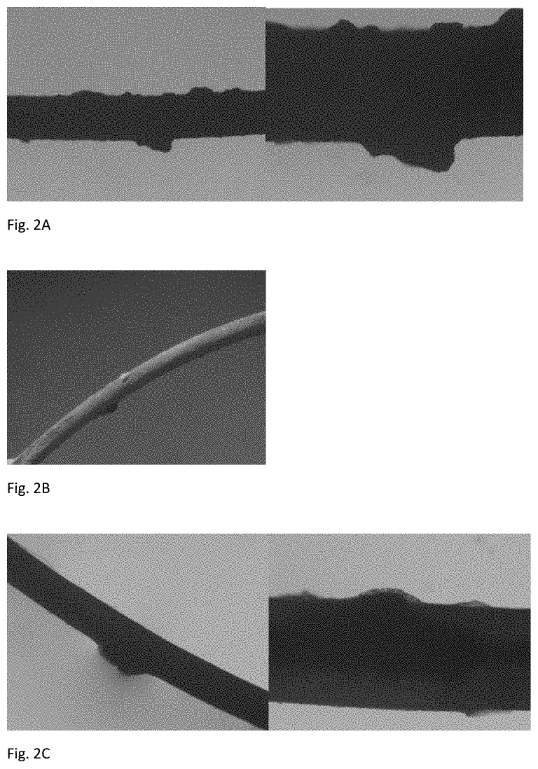

The photomicrographs shown in FIG. 2A show normal saline on the suture. After pulling this suture through the dry PCA crystals under pressure the coating was abundant and uniform coating.

A photograph of polypropylene suture coated with PCA is shown in FIG. 2B. The color is white and this photograph's very low lighting resulted in a green appearance.

FIG. 2C shows photomicrographs showing PCA on the 0 polypropylene suture under polarized light. The left photomicrograph in FIG. 2C shows low power and the right shows higher power.

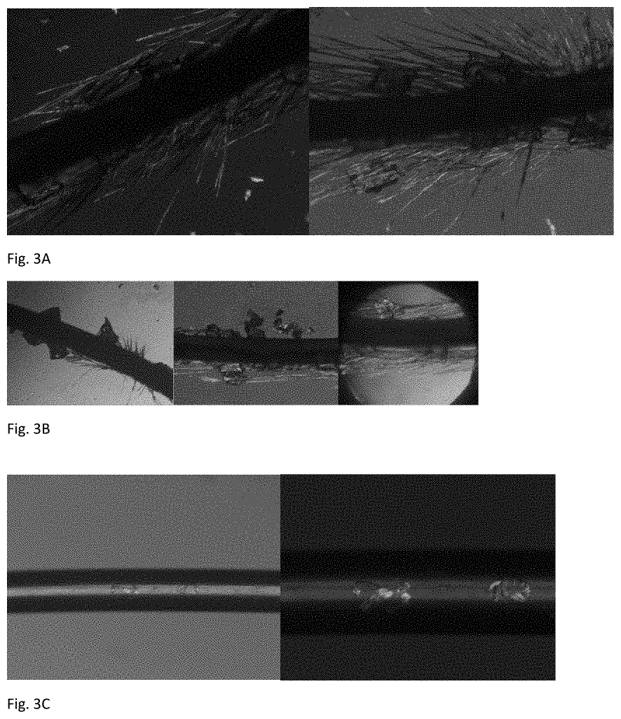

FIG. 3A shows a polarized photomicrograph of PCA crystals on 0 polypropylene suture.

FIG. 3B shows high power photomicrographs of PCA on 0 polypropylene suture. Another observation was that the suture has microscopic appearance of a hollow center which had polarized crystals as shown in FIG. 3C which shows 1% PCA in alcohol on 5 0 polypropylene suture.

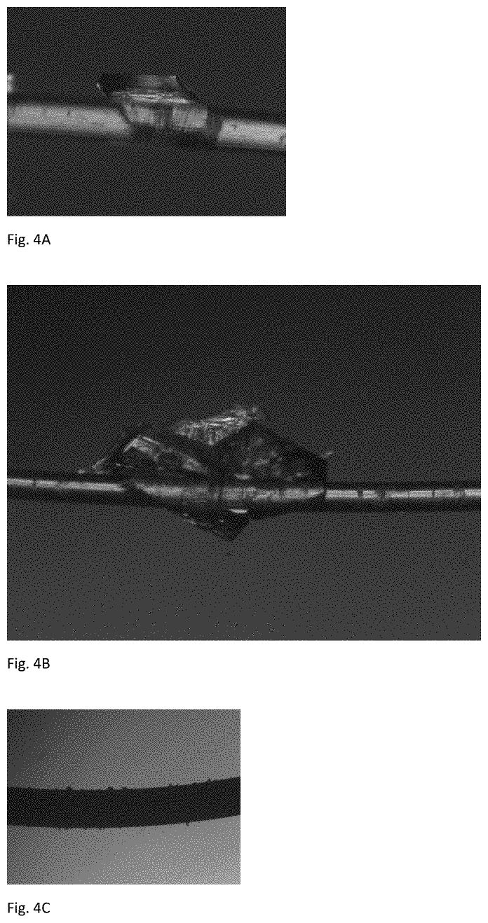

FIG. 4A shows high power polarized photomicrograph of crystal on 5-0 polypropylene from propylene glycol after 24 hours. The suture in FIG. 4A was subject to spray coating with 15% PCA in propylene glycol solution. This resulted in PCA crystal attachment with a square and/or rectangular shape.

FIG. 4B shows a high-power polarized photomicrograph. FIG. 4B shows different crystal shape and size on 5-0 polypropylene suture when in propylene glycol medium.

Nylon 0 suture was tested with dry scraping in folded paper and showed crystals attached. This resulted in visible white crystals attached to the suture surface as shown in FIG. 4C.

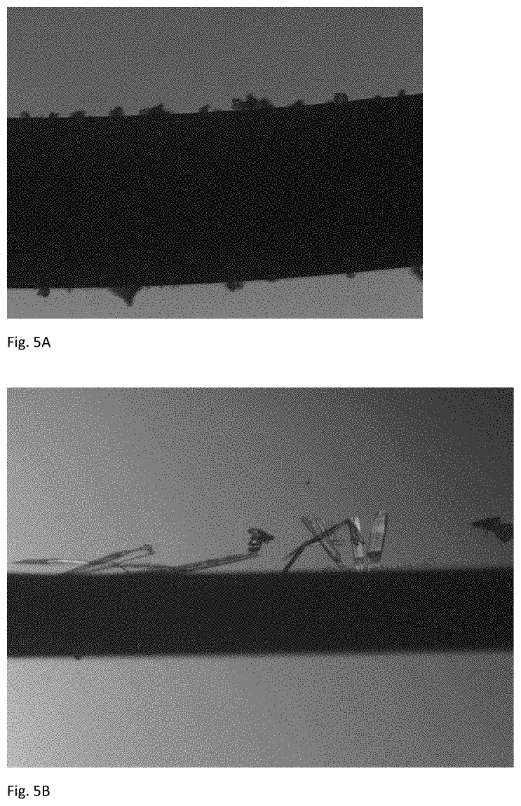

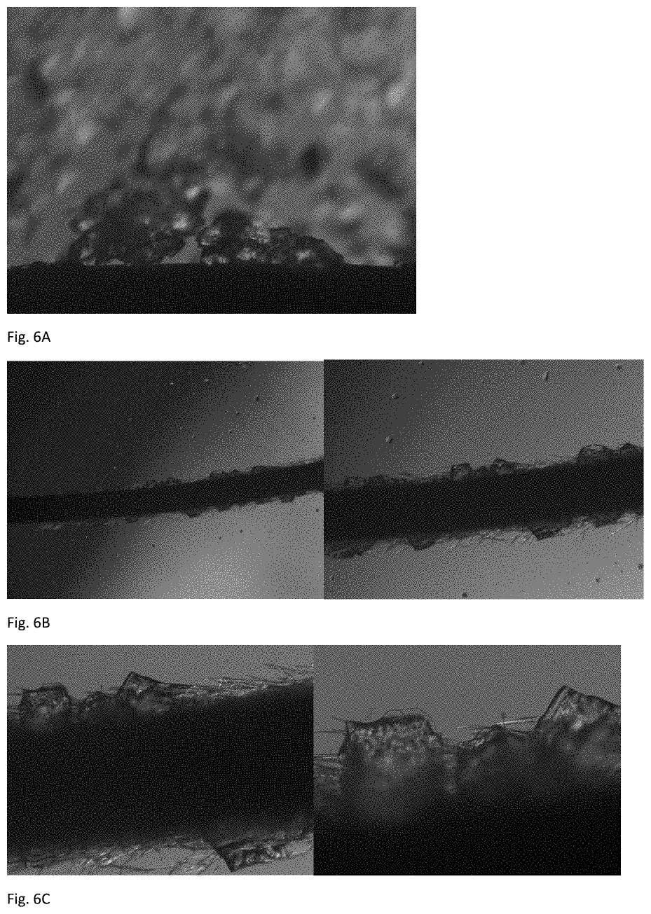

In FIG. 5A, higher power polarized light photomicrograph shows PCA crystals attached. They have the shape of dry crystals with sparse polarization reflection.

Spraying with 1% PCA in ethanol solution showed few or no crystal attachments on 0 nylon as shown in FIG. 5B.

FIG. 6A shows that testing with higher concentration of 20% PCA in ethanol showed much greater crystal adherence to 0 nylon. FIG. 6A shows an abundance of crystals in the background as well. Also, the physical shape is that of the raw PCA crystal.

In another test on 0 nylon showed different results at time zero. Soaking in 20% PCA in alcohol: negative. Dry scraping and pull through crystals: negative. Pull through with saline and raw crystals: negative.

Saline soak and 24 hours later there was coating seen on the nylon suture. Pulled through with 20% PCA and crystals showed small amount of white crystals on the suture. After 24 hours in normal saline there was an abundance of crystals when subjected to normal saline solution over-night as shown in FIG. 6B. FIG. 6B shows low power photomicrographs with polarized light showing attached crystals to 0 nylon suture. FIG. 6C shows higher power photomicrograph of polarized light with crystals on 5 0 nylon.

It was therefore confirmed that various suture materials and various gauges can be coated with PCA. The material nature was important in determining the details of the coating of the PCA. Braided polyester had no attachment in spite of the physical nature of the braid. Polypropylene was most receptive to PCA coating. Nylon was amenable to PCA coating. No tests were performed on catgut or 910 Vicryl.

The suture coating was independent of the gauge of the suture. Coating was facilitated by increased concentrations of PCA in solution. The vehicle could influence the coating as 15% PCA concentration in propylene glycol facilitated coating.

Suture coating was facilitated by heating of the solution and or suture. Suture coating was facilitated in nylon with pulling through compression of raw crystals in 20% PCA ethanol solution, but not compression application of dry crystals alone.

The braided suture was negative in all tests. The 0 nylon was positive on pull thru crystals with 20% PCA in alcohol.

Based upon these examples only polypropylene suture material would be applicable to coating at the time of surgery with a sterile PCA solution. PCA coating would be possible at time of manufacture for most sutures during the period when the suture extrusion was warm.

PCA impregnation could be accomplished at time of manufacture for bio-absorbable suture materials of all gauges. For example, this could be for an 85/15 D,L lactide/glycolide composition or a 90/10 of same. The braided suture may be suitable during the process for PCA coating of the strands.

### Examples Showing PCA Antimicrobial Effectiveness

### Example 1

Use of In Vitro Studies for Antimicrobial Susceptibility Testing of Anthocyanins, Anthocyanidins, or Metabolites and Compounds Thereof.

This example describes the method for testing the antimicrobial susceptibility of anthocyanins, anthocyanidins, or metabolites and compounds thereof. The Kirby-Bauer method of disc diffusion was used for testing, following a standard set of procedures recommended by the NCCLS. In this methodology, a set of discs saturated with either testing compounds or a control was placed on inoculated agar plates. The plates were inoculated with organisms including C. difficile, P. acnes, C. prefringens, L. casei, C. albicans, E. coli, ATTC 8739 and ATCC 43895, S. aureus, S. mutans, S. pyogenes, P. aeruginosa and K. pneumonia. The control sample was amoxicillin, an antimicrobial with very effective broad-spectrum antibiotic properties. Samples included delphinidin, pelargonidin, cyanidin CI, 28% cyanindin-3-glucoside (C3G), protocatechuic acid (PCA) and 2,4,6 Trihydroxybenzaldehyde (2,4,6 THBA).

After 18, 24, or 48 hours of incubation, depending upon the microorganism, each plate was examined. The diameters of the zones of complete inhibition were measured, including the diameter of the disc. Zones were measured to the nearest millimeter, using sliding calipers. The size of the zones of inhibition was interpreted by referring to NCCLS standard. Results were interpreted as follows: NI was no inhibition of growth under the test sample, I was inhibition of growth under the test sample, NZ indicated no zone of inhibition surrounding the test sample, and CZ indicated a clear zone of inhibition surrounding the sample and zone width in millimeters.

### Results

The testing samples had bactericidal and bacteriostatic activity against many of the organisms. Of note, P. acnes, an organism that is very difficult to treat, often requiring multiple current antibiotics for effective treatment, was susceptible to both C3G and PCA. Indeed, both of these test samples were bactericidal against P. acnes. Additionally, PCA was also effective against Staphylococcus aureus ATCC 33591, known as Methacillin Resistant Staph Aureus (MRSA).

PCA was also shown to have some effectiveness against Pseudomonas aeruginosa, a common pathogen in wounds, especially burns. Amoxicillin, the control sample, had no effect on P. aeruginosa. Similarly, Candida albicans, frequently a co pathogen in wounds, was susceptible to PCA.

In summary, the present invention provides advantages over the prior art, including providing anthocyanin, anthocyanidin, their metabolites or combinations thereof to a wound to provide a reduction or elimination of bacteria. It is contemplated that the invention will also find use in the treatment of surfaces, including medical devices and medical implants, to reduce or eliminate bacteria.

### Example 2

Use of Mouse Model to Determine Dose Levels and Intervals of Test Samples

Methods:

Mice had back skin tape stripped and the stripped site (wound) was infected with P. aeruginosa (ACTA 9027). The test reagents were applied topically in an aqueous solution on the stripped site at two hours and daily for four days.

Cyanidin 3-glucoside (C3G), an anthocyanin, and its main metabolite PCA were formulated and tested at several doses. The aqueous carrier was water. The C3G formulation included 50 mM, 100 mM and 200 mM dose concentrations. Similarly, the PCA formulation included at 50, 100 and 200 mM dose concentrations.

### Results

Results were collected from the mice at day five. Both C3G and PCA decreased the bacterial burden; however, none were statistically significant. There was a trend towards a decreasing concentration of PCA, with 50 mM being the most effective. The most effective dose of C3G was 100 mM. It is contemplated that because C3G degrades to PCA in this environment, the test results may indicate that C3G was not being tested alone, but rather was a combination of C3G and its metabolites, including a combination of C3G and PCA as the effective agents.

### Example 3

Use of Mouse Model to Further Determine Effective Dose Levels and Dose Intervals of Test Samples

Methods:

Mice had back skin tape stripped and the stripped site (wound) was infected with P. aeruginosa (ACTA 27853). The test reagents were applied topically in an aqueous solution on the stripped site at two hours and daily on day 1, 2 and 3.

C3G, an anthocyanin and its main metabolite PCA were formulated and tested at several doses. The aqueous carrier was water. The C3G formulation included 100 mM and 200 mM dose concentrations and the PCA formulation included 25 and 50 mM dose concentrations.

### Results

Results were collected from the mice at day two and four. Both C3G and PCA decreased the bacterial burden at 48 and 96 hours. The most significant decrease of bacteria was observed at 25 mM of and 100 and 200 mM of C3G. Although PCA at 25 mM reduced the bacterial burden at both time periods, its activity was statistically significant at 48 hours. C3G at both 100 mM and 200 mM significantly reduced the bacterial burden at 48 and 96 hours.

### Example 4

Use of a Mouse Model for Wound Healing

Methods:

Mice were shaved but unstrapped and uninfected (normal rodent skin). The test reagents were applied topically in an aqueous solution on the unstripped site at two hours and daily on day 1, 2 and 3.

Testing reagents consisted of C3G and PCA formulated at one dose, 100 μM in an aqueous solution.

### Results

There was little or no stimulation of IGF-1 and TGF-β at local levels observed at the 100 μM concentration of testing reagents. In fact, levels of EGF actually decreased below normal levels. There was observed a decrease of all three local growth hormones at 100 μM of C3G. These results suggest that mice skin differs in response to a dose that has been shown to stimulate human synovium to produce IGF-1. Thus, this low of a dose is not useful for rodents for this purpose.

### Example 5

Use of Mouse Model to Determine Isolated Effect of 25 mM Solution of PCA in Various Environments

Methods:

Four different conditions were used: mice had back skin tape stripped and the stripped site (wound) was infected with P. aeruginosa; mice had back skin stripped and were not infected, mice had taped stripped, infected and treated with PCA, mice were tape stripped, uninfected, and treated with PCA. When used, the PCA test reagent was applied topically in an aqueous solution on the stripped site at two hours and 24 hours.

The testing reagents consisted of and PCA formulated at one dose, 25 mM, in an aqueous solution. Levels of IGF-1, TGE-β, and EGF levels in the skin tissue at 48 hours were measured by ELISA. There were two control groups: the stripped skin and the stripped skin and infected.

### Results

The infected stripped skin showed the highest level with IGF-1 (statistically significant) and TGE-β. This is representative of tissue response to injury and infection; similarly, the EGF response was very inconsistent compared to the other two growth hormones.

The EGF response levels were different than either IGF-1 or TGE-β. They were highest in the stripped and uninfected wound and lowest in the stripped, infected and treated wound. Therefore, the treatment optimized the amount of hormone production compared to the untreated infection. This is beneficial to limit scarring while promoting healing over the controls. Overall, PCA at 25 mM acts on stripped and infected mice skin and optimizes the IGF-1 production and optimizes the local growth hormones.

### Example 6

Use of Mice to Establish Wound Promoting Effect of Compositions

Method:

Fifteen rodents were used to establish the histological findings of stripped skin, stripped and infected skin, and stripped, infected and treated wound. There were two control groups and four experimental groups according to the following:

Control Group 1: three mice with only tape stripped wounds on the back. These mice were not infected or treated. The skin was harvested at time zero, 2 and 48 hours for histology examination.

Control Group 2: three had tape stripped wounds and infection. Tissue submitted at 2 and 48 hours for histological examination.

Experimental Groups: There were 4 experimental groups. In these groups, mice had skin stripped wounds and infection. Treatment varied by reagent and dosage. Testing reagents included PCA at 25 at 25 and 50 mM and C3G at 100 and 200 mM.

Pseudomonas aeruginosa (ATCC 27853) procured from American Type Culture Collection, Manassas, Va. was used to infect the experimental groups of mice. The organism was grown overnight at 37° C. at ambient atmosphere trypticase soy agar plates supplemented with 5% sheep blood cells. The culture will be aseptically swabbed and transferred to tubes of trypticase soy broth. The optical density will be determined at 600 nm. The cultures will be diluted to provide an inoculum of approximately 9.0 log 10 CFU per mouse in a volume of 100 μL. Inoculum count was estimated before inoculation by optical density and confirmed after inoculation by dilution and back count.

The testing reagents were topically applied at 2 and 24 hours with 100 μL of fluid spread over the wound.

The following histological assessments were conducted:

Surface Cellularity: The histological assessment included the presence or absence of the surface cellularity and the depth of the cells.

Dermis:

Thickness: The thickness of the dermal layer was observed.

Hair Follicles: The hair follicles and the layer of surrounding cells were observed. Hair follicles presence is critically important to skin wound healing. (Gharzi A, Reynolds A J, Jahoda C A. Plasticity of hair follicle dermal cells in wound healing and induction. Exp Dermatol. 2003 April; 12 (2):126-36). The dermal sheath surrounding the hair follicle has the progenitor cells for contributing fibroblasts for wound healing. (Johada C A, Reynolds A J. Hair follicle dermal sheath cells: unsung participants in wound healing. Lancet. 2001 Oct. 27; 358(9291):1445-8).

Vascularity: Vascularity was observed, but an assessment of angiogenesis was not performed on the 48-hour material since new vascularity takes three to twelve days to develop. (Busuioc C J, et al. Phases of cutaneous angiogenesis process in experimental third-degree skin burns: histological and immunohistochemical study. Rom J Morphol. Embryol. 2013; 54(1):163-710.)

Inflammation: The presence of cellular infiltration was observed and its location.

Skin Thickness: The thickness of the skin was estimated related to the uninfected, untreated wound. This depth was estimated on the uniform histology photomicrographs from the surface to the muscle layer.

### Results

The following results were observed in each group:

CONTROL GROUP 1: Uninfected and untreated.

Time Zero: At time zero following the wound stripping there was cellular covering of the surface. The dermal layer was not thickened. The hair follicles have a single cellular lining. There was minimal vascularity and no inflammation. The depth of the tissue was considered zero for future benchmark. 0+

2 hours: At 2 hours following the wound stripping the surface remained covered with cellularity. The dermal layer was minimally thickened. The follicles and cellular lining were the same. There was minimal increase in vascularity and inflammation. The increase in the depth of the tissue was considered 0.5+.

48 hours: At 48 hours the wound stripped, uninfected, untreated specimens showed natural history response of surface cellular proliferation and thickness. The dermal layer was thickened. The hair follicles were present with single layer cellular lining. The vascularity was increased in amount compared to the 2-hour specimens. The inflammation was present throughout the dermis and muscle layer. The thickness was considered 0.5+.

CONTROL GROUP 2: Infected and untreated.

2 hours: The histological assessment showed the wound stripped, infected, but untreated controls at 2 hours to have multiple cellular covering on surface. There was minimal thickening of the dermal layer. The hair follicles were abundant and had double layer cellular lining. There was minimal vascularity and no inflammation in the specimens. The thickness was assigned 0.5+.

48 hours: At 48 hours the surface cellular covering was gone. The dermal layer had minimal thickening. The hair follicles were present, with minimal cellularity lining. There was marked increase in vascularity and minimal inflammation in dermis layer. The depth was considered 0.5+ compared to time zero.

Experimental Group PCA 25 Mm

48 hours: The cellular covering of the surface was abundant and multiple cell layers. The dermal layer was thickened. The hair follicles were prominent with multiple cellular lining. There was collagen proliferation between the epidermis and dermis. Additionally, there was moderate vascularity, but less than that seen in infected untreated group. There was abundant inflammation and it was greater than was seen in the PCA 50 dose. Thickness was assigned 2+.

Experimental Group PCA 50 Mm

48 hours: The surface was covered with multiple layers of cells. The dermal layer was thicker. The hair follicles had double layer of cells. There was increased vascularity. Inflammation also increased in the dermis and below the muscle layer. The tissue thickness was assigned 2+.

Experimental Group C3G 100 Mm

48 Hours: There was multiple cellular covering of the surface. The dye of the C3G was apparent on the skin surface indicating it had not changed color due to pH nor completely degraded. The dermal layer was thicker. The hair follicle had single and double cellular lining. The vascularity was prominent. There was inflammation in the dermis and muscular layer and below. The thickness of the tissue was assigned 2+.

Experimental Group C3G 200 Mm

48 Hours: There was evidence of the C3G material remaining on the skin surface. The surface cellular layer was multiple cells thick. The dermal layer was thickened. The hair follicles had single and double cellular lining. The vascularity was increased. There was inflammation in the dermis and muscular layer. The thickness was assigned 2+.

These results confirm that an anthocyanin (^{˜}38% C-3-G as the source) and the main metabolite of anthocyanins and anthocyanidins, protocatechuic acid (PCA) when applied topically at various calculated doses to the stripped skin wound of a rodent were bactericidal in 48 to 96 hours. There was a 10,000-fold kill of Pseudomonas aeruginosa in 48 hours with both reagents and dose.

The results also show by histology a simultaneous healing of the experimentally created wound in the same time frame. C-3-G and PCA in two different doses stimulated tissue repair as evidence by histology.

Specifically, the experimental model provided evidence of a histological contrast between the control and experimental groups. At 48 hours, Control Group 2 that was wound stripped and infected showed a clear contrast to the uninfected Control Group 1. In the skin stripped infected group there was loss of the epithelial cellular covering, no follicular cellular proliferation, marked increase in vascularity and little inflammatory response. This histological condition provided clear contrast to the treatment groups. All treatment groups by comparison showed healing response with multiple layer cellular proliferation on the surface, multiple layer cellular proliferation along the hair follicles, less vascularity, but an inflammatory cellular response in the dermis and muscular levels. PCA at a concentration of 25 mM also showed collagen layer formation between the epidermis and dermis. This response is beneficial in the use of anthocyanin and anthocyanidins and metabolites thereof as a cosmetic agent to promote wound healing and improve skin health, including wrinkle reduction or removal. This method of use of anthocyanin and anthocyanidin metabolites, and particularly PCA, is based upon the two-fold response; the collagen layer increase and the skin swelling that increased the depth of the skin.

While this disclosure includes specific examples, it will be apparent after an understanding of the disclosure of this application has been attained that various changes in form and details may be made in these examples without departing from the spirit and scope of the claims and their equivalents.

1. A suture or surgical staple comprising protocatechuic acid, wherein the protocatechuic acid is coated on, or impregnated in, the suture or surgical staple.

2. (canceled)

3. (canceled)

4. The suture or surgical staple of claim 1, wherein the suture or surgical staple comprises polypropylene.

5. The suture or surgical staple of claim 1, wherein the suture or surgical staple comprises 85/15 D,L lactide/glycolide.

6. The suture or surgical staple of claim 1, wherein the suture or surgical staple comprises nylon.

7. The suture Or of claim 1, wherein the suture or surgical staple comprises polyester and/or braided polyester.

8. The suture or of claim 1, wherein the suture or surgical staple comprises catgut.

9. The suture or surgical staple of claim 1, wherein the protocatechuic acid coats 25% or more of the surface of the suture or surgical staple.

10. The suture or surgical staple of claim 1, wherein the protocatechuic acid coats 75% or more of the surface of the suture or surgical staple.

11. The suture or surgical staple of claim 1, wherein the protocatechuic acid coats 95% or more of the surface of the suture or surgical staple.

12. The suture or surgical staple of claim 1, wherein the protocatechuic acid has a purity greater than 95%.

13. A method of making a suture of claim 1 comprising:
placing a suture in contact with protocatechuic acid.

14. The method of claim 13, wherein the suture is drawn through dry protocatechuic acid.

15. The method of claim 14, wherein the suture is drawn through dry protocatechuic acid under pressure.

16. The method of claim 13, wherein the protocatechuic acid comprises protocatechuic acid crystals.

17. The method of claim 13, wherein the suture comprises polypropylene.

18. The method of claim 13, wherein the suture comprises nylon.

19. The method of claim 13, wherein the suture comprises polyester and/or braided polyester.

20. The method of claim 13, wherein the suture comprises catgut.

21. The method of claim 13, wherein the suture comprises 85/15 D,L lactide/glycolide.

22. The suture of claim 1, wherein the suture size 0 or 5/0.

23. The suture of claim 1, wherein the suture comprises catgut, copolymer of 10% L lactide and 90% glycolide, polyglycolic acid, polylactic acid, poliglecaprone 25, polydioxanone, nylon, polyester, braided polyester, Polyvinylidene fluoride, Poly(lactide-co-glycolide) (PLGA) 85/15 D,L lactide/glycolide, Poly(lactide-co-glycolide) (PLGA) 90:10 glycolide:l-lactide, and/or polypropylene.
